(19) United States
(12) Patent Application Publication
(10) Pub. No.: US 2009/0053287 A1
(43) Pub. Date: Feb. 26, 2009

(54) POROUS DRESSING

(75) Inventors: Ching-Cheng HUANG, Taipei Hsien (TW); Quang-Wei CHANG, Taipei Hsien (TW); Yung-Sheng LIN, Changhua County (TW); Ting-Kai LEUNG, Taipei City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignees: Medical And Pharmaceutical Industry Technology And Development Center, Taipei Hsien (TW); National Applied Research Laboratories, Taipei (TW); Taipei Medical University, Taipei (TW)

(21) Appl. No.: 11/926,575

(22) Filed: Oct. 29, 2007

(30) Foreign Application Priority Data
Aug. 23, 2007 (TW) 096129302

(51) Int. Cl. A61L 15/00

(52) U.S. Cl. 424/445

(57) ABSTRACT

A porous dressing is provided. The porous dressing includes a polymeric layer, a pharmaceutically active ingredient and a metal oxide. The polymeric layer has a porosity and a bio-compatibility, and the pharmaceutically active ingredient and the metal oxide distribute in one selected from a group consisting of in the polymeric layer, on a surface of the polymeric layer and a combination thereof.

### FIELD OF THE INVENTION

The present invention relates to a porous dressing, and more particularly to a porous dressing comprising metal oxide that improves the additional value thereof.

### BACKGROUND OF THE INVENTION

In order to provide comfort and convenience, most dressings in the market have porosity, water-proof and air permeability. Thus, many newly developed bio-materials having such properties are used in the market of dressing. The bio-material is derived from a natural source or a synthetic material, which has bio-capability and can be implanted into or combined with an in vivo system for replacing or repairing a part of the in vivo system, or the bio-material can contact directly with a living body for executing the vital function. For example, the Taiwan patent No. I247614 discloses a wound dressing having a strengthening function, which provides a space for cell growth by using a bio-capable material.

Besides the fastidious material of the dressing, many dressings even have an anti-bacteria function for preventing the attack of the foreign bacteria. In both of the above-mentioned Taiwan patent No. I247614 and Taiwan patent publication No. 00590763, a dressing with a doped nano gold or nano silver for functions of anti-bacteria and promoting the growth rate of cells is disclosed.

In view of the drawbacks of current techniques, the inventors develop a porous dressing that replaces the nano gold or nano silver with metal oxide. The dressing not only has a lower cost and various advantages of the prior arts, but contains an undisclosed function that enables the drug in the dressing to be absorbed efficiently. The summary of the present invention is described below.

### SUMMARY OF THE INVENTION

The present invention provides a porous dressing comprising a metal oxide, which has a lower cost than the prior art. Furthermore, the present invention has an unexpected function owing to the adding of the metal oxide, for example, the porous dressing of the present invention can improve the absorbability of the drug, improve blood circulation, activate metabolism, promote tissue regeneration and activate the immune system, etc.

It is an aspect of the present invention to provide a porous dressing, which comprises a polymeric layer having a porosity and a bio-compatibility, a pharmaceutically active ingredient and a metal oxide, wherein the pharmaceutically active ingredient and the metal oxide distribute in one selected from a group consisting of in the polymeric layer, on a surface of the polymeric layer and a combination thereof.

Preferably, the polymeric layer has an elasticity, an extensibility and a 3D porous structure with a plurality of pores connected with each other and is bio-degradable and prepared by a lyophilization.

Preferably, the polymeric layer is made of at least one selected from a group consisting of a chitosan, a sodium alginate, a cellulose, a hyaluronic acid, a collagen, a polyurethane, a gel, a polylactic acid, a polygiycolic acid, a poly(lactic-co-glycolic acid), a poly(lactic-co-aminocaproic acid), a poly(3-hydroxybutyrate) and a poly(3-hydroxybutyrate-co-3-hydroxyvalerate).

Preferably, the pharmaceutically active ingredient is a nonsteroidal anti-inflammatory drug (NSAID).

Preferably, the metal oxide comprises at least one selected from a group consisting of an aluminum oxide, a magnesium oxide and a ferric oxide.

Preferably, the metal oxide comprises 60-95% aluminum oxide, 1-10% magnesium oxide and 1-20% ferric oxide.

Preferably, the porous dressing further comprises a cohesive layer.

It is another aspect of the present invention to provide a porous dressing comprising a polymer and a metal oxide, wherein the polymer has a porosity and a bio-compatibility, and the metal oxide distributes in one selected from a group consisting of in the polymeric layer, on a surface of the polymeric layer and a combination thereof.

According to the present invention, the porous dressing further comprises a pharmaceutically active ingredient.

Preferably, the pharmaceutically active ingredient is a nonsteroidal anti-inflammatory drug (NSAID).

Preferably, the porous dressing further comprises a cohesive layer.

Preferably, the polymer is water-proof and has a humidity permeability.

Preferably, the polymer has an elasticity, an extensibility and a 3D porous structure with a plurality of pores connected with each other and is biodegradable and prepared by a lyophilization.

Preferably, the polymer is made of at least one selected from a group consisting of a chitosan, a sodium alginate, a cellulose, a hyaluronic acid, a collagen, a polyurethane, a gel, a polylactic acid, a polygiycolic acid, a poly(lactic-co-glycolic acid), a poly(lactic-co-aminocaproic acid), a poly(3-hydroxybutyrate) and a poly(3-hydroxybutyrate-co-3-hydroxyvalerate).

Preferably, the metal oxide comprises at least one selected from a group consisting of an aluminum oxide, a magnesium oxide and a ferric oxide.

Preferably, the metal oxide comprises 60-95% aluminum oxide, 1-10% magnesium oxide and 1-20% ferric oxide.

It is a further aspect of the present invention to provide a medical medium for a wound care, which comprises a base having a porous structure and a metal oxide distributed in one selected from a group consisting of in the base, on a surface of the base and a combination thereof. According to the present invention, the base covers a wound-bearing skin of a subject and the metal oxide activates a water molecule resonance effect in the subject.

Preferably, the base is a polymer.

Preferably, the medical medium is selected from a group consisting of a filler material, a burn dressing and a drug delivery system.

Preferably, the medical medium further comprises a pharmaceutically active ingredient.

Other objects, advantages and efficacies of the present invention will be described in detail below taken from the preferred embodiments with reference to the accompanying drawings, in which:

### BRIEF DESCRIPTION OF THE DRAWINGS

FIG. 1 is a diagram showing the porous dressing of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

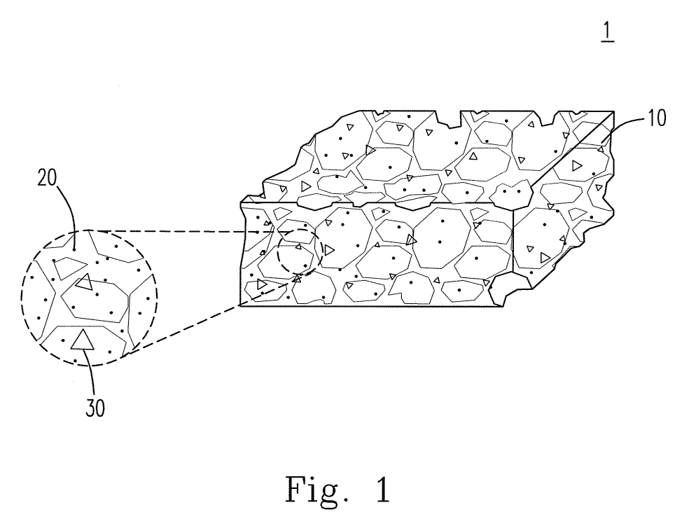

Please refer to FIG. 1, which is a diagram showing the porous dressing of the present invention. As FIG. 1 shows, the porous dressing 1 comprises a polymeric layer 10 having porosity, bio-compatibility, bio-degradability, water-proof and humidity permeability. The porosity herein means that the polymeric layer 10 has a 3D porous structure with a plurality of pores connected with each other to provide a better ventilation. In order to make the porous dressing 1 more comfortable and be applied to joints, the polymeric layer 10 is preferably prepared by an elastic and extendable material. The polymeric layer 10 is made of at least one selected from a group consisting of chitosan, sodium alginate, cellulose, hyaluronic acid, collagen, polyurethane, gel, polylactic acid, polygiycolic acid, poly(lactic-co-glycolic acid), poly(lactic-co-aminocaproic acid), poly(3-hydroxybutyrate) and poly(3-hydroxybutyrate-co-3-hydroxyvalerate).

Please continually refer to FIG. 1. The porous dressing 1 further comprises a metal oxide 20 derived from the natural mineral, which comprises at least one selected from a group consisting of an aluminum oxide, a magnesium oxide and a ferric oxide. The other ingredients of the metal oxide 20 include titanium dioxide, titanium boride and more natural minerals, for instance, silicon oxide, zinc hydroxide and carbide. Preferably, the metal oxide 20 at least includes 60-95% aluminum oxide, 1-20% ferric oxide and 1-10% magnesium oxide. Furthermore, the metal oxide of the present invention has an anti-bacterial rate of over 99.9% against Staphylococcus and Escherichia coli according to the AATCC100 standard. Additionally, the metal oxide releases negative ion and will not release free irradiation that is generally considered as a danger causing a genetic mutation and cancer. Hence, it is understood that although the present invention is a product contacting with the human body, the users will be healthy and safe.

Please continually refer to FIG. 1. The porous dressing 1 of the present invention further comprises a pharmaceutically active ingredient 30 according to actual needs, wherein the pharmaceutically active ingredient 30 distributes in one selected from a group consisting of in the polymeric layer 10, on a surface of the polymeric layer 10 and a combination thereof. According to actual needs, the pharmaceutically active ingredient 30 is an antibiotics, an anti-inflammation drug or a drug for tissue regeneration, etc. The metal oxide 20 will enhance the absorption of the pharmaceutically active ingredient 30 in the human body. Additionally, the metal oxide 20 of the present invention activates a water molecule resonance effect in the human body, and thus the water molecule becomes a smaller one. As a result, the oxygen content is elevated, and the resonated water molecule will slightly increase the subcutaneous temperature, dilate the blood vessels, decrease the viscosity and tension of the blood and accelerate the blood stream. Accordingly, the water molecule resonance effect improves the microcirculation in the human body, improves the wound healing and eases off the joint pain. Therefore, the porous dressing 1 of the present invention still has above effects even in the absence of any pharmaceutically active ingredient 30.

### Example I

### Preparation of the Porous Dressing by Using Chitosan

A proper amount of chitosan with over eighty five percent of deacetylation is dissolved in a solution containing 2%-5% acetic acid for preparing a 2% (w/w) polymer solution. The polymer solution is filled into a ferric plate with a minimum amount for covering the bottom of the plate to obtain a sample, and the height of the filling volume is controlled in accordance with the depth of the sample. Subsequently, the sample is lyophilized at −35° C. to −40° C. for 24 hrs and immersed in 1N NaOH for 1-5 minutes followed by several washes with ultra pure water for continuing the preparation under neutral condition (pH 7.4, 25° C.). Finally, in order to remove unnecessary water, the sample is lyophilized again and then a porous chitosan dressing is obtained. In the preparation, the polymer solution can be replaced with any amine aquatic solution that has bio-degradability and bio-compatibility.

### Example II

### Preparation of the Porous Dressing Comprising a Metal Oxide by Using Chitosan

A polymer solution is prepared for use by the method described in the first preferred embodiment. Since the metal oxide does not dissolve in an aquatic solution, the surfactant span-80 is mixed with the metal oxide in a 4:1 ratio to prepare a metal oxide emulsion. Subsequently, the metal oxide emulsion is mixed evenly with the polymer solution in an 1:10 ratio. The mixed solution is filled into a ferric plate with a minimum amount for covering the bottom of the plate to obtain a sample, and the height of the filling volume is controlled in accordance with the depth of the sample. Then, the sample is lyophilized at −35° C. to −40° C. for 24 hrs and immersed in 1N NaOH for 1-5 minutes followed by several washes with ultra pure water for continuing the preparation under neutral condition (pH 7.4, 25° C.). Finally, in order to remove unnecessary water, the sample is further lyophilized and then a porous chitosan dressing comprising a metal oxide is obtained. In the preparation, the polymer solution can be replaced with any amine aquatic solution that has bio-degradability and bio-compatibility. Additionally, it is acceptable to decrease the ratio of surfactant span-80 mixed with the metal oxide for the metal oxide emulsion preparation from 4:1 to 1:1.

### Example III

### Preparation of the Porous Dressing Comprising Indomethacin by Using Chitosan

A polymer solution is prepared for use by the method described in the first preferred embodiment. Since the nonsteroidal anti-inflammatory, Indomethacin (IDM) does not dissolve in an aquatic solution, the surfactant span 80 is mixed with the IDM in a 4:1 ratio to prepare an IDM emulsion. Subsequently, the IDM emulsion is mixed evenly with the polymer solution in an 1:10 ratio. The mixed solution is filled into a ferric plate with a minimum amount for covering the bottom of the plate to obtain a sample, and the height of the filling volume is controlled in accordance with the depth of the sample. Then, the sample is lyophilized at −35° C. to −40° C. for 24 hrs and immersed in 1N NaOH for 1-5 minutes followed by several washes with ultra pure water for continuing the preparation under neutral condition (pH 7.4, 25° C.). Finally, in order to remove unnecessary water, the sample is further lyophilized and then a porous chitosan dressing comprising IDM is obtained. In the preparation, the polymer solution can be replaced with any amine aquatic solution that has bio-degradability and bio-compatibility. Additionally, it is acceptable to decrease the ratio of surfactant span-80 mixed with IDM for the IDM emulsion preparation from 4:1 to 1:1

### Example IV

### Preparation of the Porous Dressing Comprising Indomethacin and Metal Oxide by Using Chitosan

An IDM emulsion and a polymer solution are prepared by the method described in the third preferred embodiment, and a little amount of metal oxide is mixed and emulsified evenly with the IDM emulsion in an 1:10 ratio repeatedly. Subsequently, the IDM emulsion is mixed evenly with the polymer solution in an 1:10 ratio. The following steps are the same as the second preferred embodiment and not described here. In the preparation, it is acceptable to increase the ratio of metal oxide mixed with IDM emulsion from 1:10 to 1:1.

### Example V

### Preparation of the Porous Dressing by Using Sodium Alginate

A proper amount of sodium alginate is dissolved in the ultra pure water for preparing an 1%˜2% (w/w) polymer solution. The polymer solution is filled into a ferric plate with a minimum amount for covering the bottom of the plate to obtain a sample, and the height of the filling volume is controlled in accordance with the depth of the sample. Subsequently, the sample is lyophilized at −35° C. to −40° C. for 24 hrs. After lyophilization, the sample is immersed into a 0.02 M˜0.2 M calcium chloride solution by an immersion method to form a calcified alginate sponge, thereby finishing the preparation of the porous sodium alginate dressing. In the preparation, the polymer solution can be replaced with any aquatic solution with an OH group that has bio-degradability and bio-compatibility.

### Example VI

### Preparation of the Porous Dressing Comprising a Metal Oxide by Using Sodium Alginate

A polymer solution is prepared for use by the method described in the fifth preferred embodiment. Since the metal oxide does not dissolve in an aquatic solution, the surfactant span-80 is mixed with the metal oxide in a 4:1 ratio to prepare a metal oxide emulsion. Subsequently, the metal oxide emulsion is mixed evenly with the polymer solution in an 1:10 ratio. The mixed solution is filled into a ferric plate with a minimum amount for covering the bottom of the plate to obtain a sample, and the height of the filling volume is controlled in accordance with the depth of the sample. Then, the sample is lyophilized at −35° C. to −40° C. for 24 hrs. After lyophilization, the sample is immersed into a 0.02 M˜0.2 M calcium chloride solution by an immersion method to form a calcified alginate sponge, thereby finishing the preparation of the porous sodium alginate dressing comprising a metal oxide. In the preparation, the polymer solution can be replaced with any aquatic solution with a OH group that has bio-degradability and bio-compatibility. Additionally, it is acceptable to decrease the ratio of surfactant span-80 mixed with metal oxide for the metal oxide emulsion preparation from 4:1 to 1:1.

### Example VII

### Preparation of the Porous Dressing Comprising Indomethacin by Using Sodium Alginate

A polymer solution is prepared for use by the method described in the fifth preferred embodiment. Since the nonsteroidal anti-inflammatory, Indomethacin (IDM) does not dissolve in an aquatic solution, the surfactant span 80 is mixed with the IDM in a 4:1 ratio to prepare an IDM emulsion. Subsequently, the IDM emulsion is mixed evenly with the polymer solution in an 1:10 ratio. The mixed solution is filled into a ferric plate with a minimum amount for covering the bottom of the plate to obtain a sample, and the height of the filling volume is controlled in accordance with the depth of the sample. Then, the sample is lyophilized at −35° C. to −40° C. for 24 hrs. After lyophilization, the sample is immersed into a 0.02 M˜0.2 M calcium chloride solution by an immersion method to form a calcified alginate sponge, thereby finishing the preparation of the porous sodium alginate dressing comprising IDM. In the preparation, the polymer solution can be replaced with any aquatic solution with an OH group that has bio-degradability and bio-compatibility. Additionally, it is acceptable to decrease the ratio of surfactant span-80 mixed with IDM for the IDM emulsion preparation from 4:1 to 1:1.

### Example VIII

### Preparation of the Porous Dressing Comprising Indomethacin and Metal Oxide by Using Sodium Alginate

An IDM emulsion and a polymer solution are prepared by the method described in the seventh preferred embodiment, and a little amount of metal oxide is mixed and emulsified evenly with the IDM emulsion in an 1:10 ratio repeatedly. Subsequently, the IDM emulsion is mixed evenly with the polymer solution in an 1:10 ratio. The following steps are the same as the seventh preferred embodiment and not described here. In the preparation, it is acceptable to increase the ratio of metal oxide mixed with IDM emulsion from 1:10 to 1:1.

### Example IX

### Preparation of the Porous Dressing Including a Cohesive Layer

A porous dressing is prepared by the method described in the first to the eighth preferred embodiments, and any one surface thereof is covered by a layer of adhesive, which is selected from a group consisting of acrylic adhesive, epoxy resin adhesive and hot melt glue.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A porous dressing, comprising:
a polymeric layer having a porosity and a bio-compatibility;
a pharmaceutically active ingredient distributed in one selected from a group consisting of in the polymeric layer, on a surface of the polymeric layer and a combination thereof; and
a metal oxide distributed in one selected from a group consisting of in the polymeric layer, on a surface of the polymeric layer and a combination thereof.

2. A porous dressing as claimed in claim 1, wherein the polymeric layer has an elasticity, an extensibility and a 3D porous structure with a plurality of pores connected with each other and is bio-degradable and prepared by a lyophilization.

3. A porous dressing as claimed in claim 1, wherein the polymeric layer is made of at least one selected from a group consisting of a chitosan, a sodium alginate, a cellulose, a hyaluronic acid, a collagen, a polyurethane, a gel, a polylactic acid, a polygiycolic acid, a poly(lactic-co-glycolic acid), a poly(lactic-co-aminocaproic acid), a poly(3-hydroxybutyrate) and a poly(3-hydroxybutyrate-co-3-hydroxyvalerate).

4. A porous dressing as claimed in claim 1, wherein the pharmaceutically active ingredient is a nonsteroidal anti-inflammatory drug (NSAID).

5. A porous dressing as claimed in claim 1, wherein the metal oxide comprises at least one selected from a group consisting of an aluminum oxide, a magnesium oxide and a ferric oxide.

6. A porous dressing as claimed in claim 5, wherein the metal oxide comprises 60-95% aluminum oxide, 1-10% magnesium oxide and 1-20% ferric oxide.

7. A porous dressing as claimed in claim 1, further comprising a cohesive layer.

8. A porous dressing, comprising:
a polymer having a porosity and a bio-compatibility; and
a metal oxide distributed in one selected from a group consisting of in the polymeric layer, on a surface of the polymeric layer and a combination thereof.

9. A porous dressing as claimed in claim 8, further comprising a pharmaceutically active ingredient.

10. A porous dressing as claimed in claim 9, wherein the pharmaceutically active ingredient is a nonsteroidal anti-inflammatory drug (NSAID).

11. A porous dressing as claimed in claim 8, further comprising a cohesive layer.

12. A porous dressing as claimed in claim 8, wherein the polymer is water-proof and has a humidity permeability.

13. A porous dressing as claimed in claim 8, wherein the polymer has an elasticity, an extensibility and a 3D porous structure with a plurality of pores connected with each other and is biodegradable and prepared by a lyophilization.

14. A porous dressing as claimed in claim 8, wherein the polymer is made of at least one selected from a group consisting of a chitosan, a sodium alginate, a cellulose, a hyaluronic acid, a collagen, a polyurethane, a gel, a polylactic acid, a polygiycolic acid, a poly(lactic-co-glycolic acid), a poly(lactic-co-aminocaproic acid), a poly(3-hydroxybutyrate) and a poly(3-hydroxybutyrate-co-3-hydroxyvalerate).

15. A porous dressing as claimed in claim 8, wherein the metal oxide comprises at least one selected from a group consisting of an aluminum oxide, a magnesium oxide and a ferric oxide.

16. A porous dressing as claimed in claim 15, wherein the metal oxide comprises 60-95% aluminum oxide, 1-10% magnesium oxide and 1-20% ferric oxide.

17. A medical medium for a wound care, comprising:
a base having a porous structure; and
a metal oxide distributed in one selected from a group consisting of in the base, on a surface of the base and a combination thereof, wherein the base covers a wound-bearing skin of a subject and the metal oxide activates a water molecule resonance effect in the subject.

18. A medical medium as claimed in claim 17, wherein the base is a polymer.

19. A medical medium as claimed in claim 17 being selected from a group consisting of a filler material, a burn dressing and a drug delivery system.

20. A medical medium as claimed in claim 17, further comprising a pharmaceutically active ingredient.
